(19) United States
(12) Patent Application Publication
(10) Pub. No.: US 2015/0107586 A1
(43) Pub. Date: Apr. 23, 2015

(54) Quick-Release Coupling Structure Between Gas Delivery Tube and Respiratory Mask

(71) Applicant: Yen-Ting Kuo, Tsai Kung Village (TW)

(72) Inventor: Yen-Ting Kuo, Tsai Kung Village (TW)

(21) Appl. No.: 14/060,664

(22) Filed: Oct. 23, 2013

(51) Int. Cl. A61M 16/08; A61M 16/06

(52) U.S. Cl. CPC A61M 16/0816; A61M 16/0875; A61M 16/06
USPC 128/202.27

(57) ABSTRACT

A quick-release coupling structure between a gas delivery tube and a respiratory mask includes a connecting member assembled to the respiratory mask and a pipe-end joint detachably coupled to the connecting member. The connecting member has two bendable tongues each having a protuberance formed on an outer surface thereof. The pipe-end joint has two sockets for receiving the tongues, and the sockets are provided with resilient members that press upon the tongues and can resiliently retract. The pipe-end joint has two retaining recesses at the sockets for retaining the protuberances. When the pipe-end joint is combined with the connecting member, the protuberances are retained by the retaining recesses. When the resilient members are pressed inward, the tongues retract and the protuberances leave the retaining recesses, so the pipe-end joint can be easily detached. Thereby, easy attachment and detachment between the gas delivery tube and the respiratory mask can be achieved.

### BACKGROUND OF THE INVENTION

1. Technical Field

The present invention relates to respiratory masks, and more particularly to a quick-release coupling structure that connects a gas delivery tube to a respiratory mask. The disclosed structure has a connecting member on the respiratory mask and a pipe-end joint on the air delivery pipe, which can be easily coupled together by insertion and can be easily separated by slightly pressing resilient members at two sides of the pipe-end joint.

2. Description of Related Art

Snoring is caused by upper respiratory tract obstruction, and may be the consequence of obesity, aging or upper respiratory tract stricture. When sleeping, people who snore have their throat muscles relax and lead to airway collapse. As a result, air cannot enter lungs smoothly, and hypoxemia happens. At this time, the brain, in response to hypoxia, orders the body to breathe hard. Hard breathing thus briefly wakes snoring people and prevents the deep sleep stage. Such apnea may happen tens or even more than one hundred times in a single night. This so-called “Sleep Apnea Syndrome” can destroy the patients' sleep quality with middle or maintenance insomnia, making the patients wake up with headaches, feel lethargic or doze off during daytime and indirectly lead to attention deficit, memory deterioration, dysphoria, character changes, and libido decrease. The patients may suffer pollakisuria in additional to snoring, and in an even worse case, these conditions can induce or aggravate cardiovascular diseases.

Mild sleep apnea syndrome may be improved if the patient takes regular exercise, loses some weight, sleep on his/her side, keep off drinking and taking hypnagogue before sleep and/or wear a stop snoring device (e.g. a mouthpieces). For serious sleep apnea syndrome, surgery or non-invasive therapeutic devices such as positive pressure ventilators may be needed. Particularly, when working with a respiratory mask, a positive pressure ventilator can provide a more-than-90% success rate. This treatment uses pressure generated by a respirator to support the impotent breathing passage of a patient wearing the connected respiratory mask, thereby maintaining the patient's breathing passage to be unobstructed.

However, besides some disadvantages about the existing respiratory masks and ventilators that make patients less willing to receive the treatment, including air leakage, mask-induced constriction, expiratory resistance, pipe weight, dry air and non-portability, post-use cleanness of the respiratory mask is also troublesome. For example, Taiwan Patent No. 1377073 titled “Respiratory Mask” discloses a respiratory mask having a connecting hole formed at its front end for allowing an L-shaped connecting member associated with a gas delivery tube to be inserted and held therein. The connecting member has one of its ends associated with the air delivery pipe, as described above. The opposite end of the connecting member inserted into the connecting hole has a tubular structure. The tubular structure is terminated with petals forming an abutting flange that is diametrically slightly greater than the inner wall of the connecting hole. Thereby, when the tubular structure of the connecting member is inserted into the connecting hole, the abutting flange abuts against the end wall of the connecting hole, so that the connecting member is allowed to freely rotate in the connecting hole yet secured from coming off.

In addition to the foregoing Taiwan Patent, there are some more prior patents adopt similar technical schemes to provide a rotatable connecting member that allows the associated gas delivery tube to move with the respiratory mask when the wearer turns his/her head, such as China Patent No. CN102046233 and U.S. Pat. No. D652,913 and U.S. Pat. No. 8,225,793. While these designs are successful in preventing the gas delivery tube from winding around the wearer's neck and thereby ensuring comfortable and airtight wear of the respiratory mask, none of them take disassembly into account. To specify, when the connecting member needs to be detached from the respiratory mask for the purpose of cleanness, the abutting flange of the connecting member forced into the connecting hole to form a force fit connection has to be pulled out with a large bias pulling force. This causes the detachment to be difficult to users, particularly to weak users. In addition, after frequent and repeated attachment and detachment, the connecting member tends to get worn at its connecting components, leading to unsecured connection, air leakage and even breakage.

### SUMMARY OF THE INVENTION

For improving the conventional respiratory masks by providing more convenient attachment and detachment between the gas delivery tube and the pipe-end joint, the present invention herein provides a quick-release coupling structure that comprises a connecting member that is rotatably assembled to the respiratory mask and has two tongues having oppositely arranged protuberances, and further comprises a pipe-end joint that is connected to the gas delivery tube and has therein sockets for receiving the tongues, so that the protuberances are retained by retaining recesses provided in the sockets of the pipe-end joint. The pipe-end joint has the sockets provided with resilient members that press upon the tongues and are resiliently retract inward when receiving a pressing force. With the configuration described above, a user can easily attach or detach the pipe-end joint to or from the respiratory mask by using his/her fingers to simultaneously press the resilient members.

The technical scheme the present invention implements to address the technical problem is using connecting member that is rotatably assembled to a front end of a respiratory mask, and a pipe-end joint that is connected to the gas delivery tube and configured to be detachably coupled to the connecting member. The connecting member having two elastic, bendable tongues that are provided at two opposite outer edges of the connecting member and each have a protuberance formed on an outer surface thereof. The pipe-end joint has two sockets formed inside an end thereof facing the connecting member, in which the two sockets are configured to receive the two tongues, respectively. The pipe-end joint has two resilient members that are configured to press upon the tongues inserted in the sockets and are able to resiliently retract inward the sockets when receiving a pressing force. The pipe-end joint further has two retaining recesses that are configured to retain the two protuberances on the two tongues that are inserted in the sockets, respectively.

In the foregoing structure, the connecting member further has two quasi-semicircular raised portions formed at two other opposite outer edges thereof, and the pipe-end joint correspondingly has a groove for receiving the two raised portions, respectively.

In the foregoing structure, the pipe-end joint has two guiding stages and the two sockets are formed between ends of the guiding stages so that the tongues that are not well aligned with the sockets are allowed to move along the guiding stages until falling into the sockets.

The beneficial effects of the present invention is that a connecting member assembled to the respiratory has two tongues having oppositely arranged protuberances, and the tongues are to be inserted into sockets inside a pipe-end joint, so that the protuberances are retained by retaining recesses provided in the sockets of the pipe-end joint. The pipe-end joint has the sockets provided with resilient members that press upon the tongues and are resiliently retract inward when receiving a pressing force. With the configuration described above, a user can easily attach or detach the pipe-end joint to or from the respiratory mask by using his/her fingers to slightly press the resilient members to make the two tongues move inward. As a result, the protuberances on the tongues are withdrawn from the retaining recesses, so that the connecting member can be easily removed, achieving convenient attachment and detachment.

### BRIEF DESCRIPTION OF THE DRAWINGS

The invention as well as a preferred mode of use, further objectives and advantages thereof will be best understood by reference to the following detailed description of an illustrative embodiment when read in conjunction with the accompanying drawings, wherein:

FIG. 1 is a perspective view of the present invention;

FIG. 2 is an exploded view of the present invention;

FIG. 3 is a perspective view of the present invention taken from a different viewpoint;

FIG. 4 is a cross-sectional view of the pipe-end joint and the connecting member of the present invention;

FIG. 5 is a top-down cross-sectional view of the pipe-end joint and the connecting member of the present invention; and

FIG. 6 illustrates detachment of the pipe-end joint from the connecting member according to the present invention.

### DETAILED DESCRIPTION OF THE INVENTION

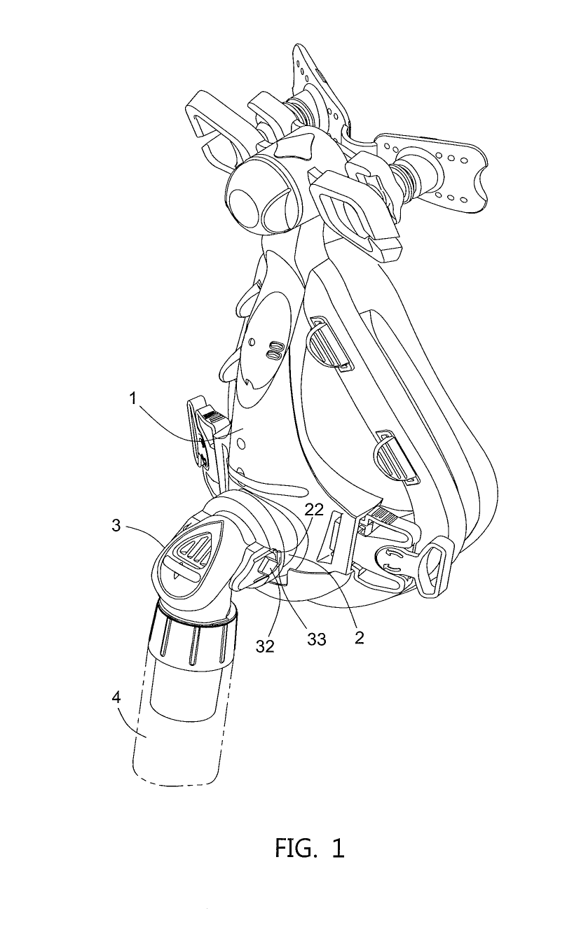

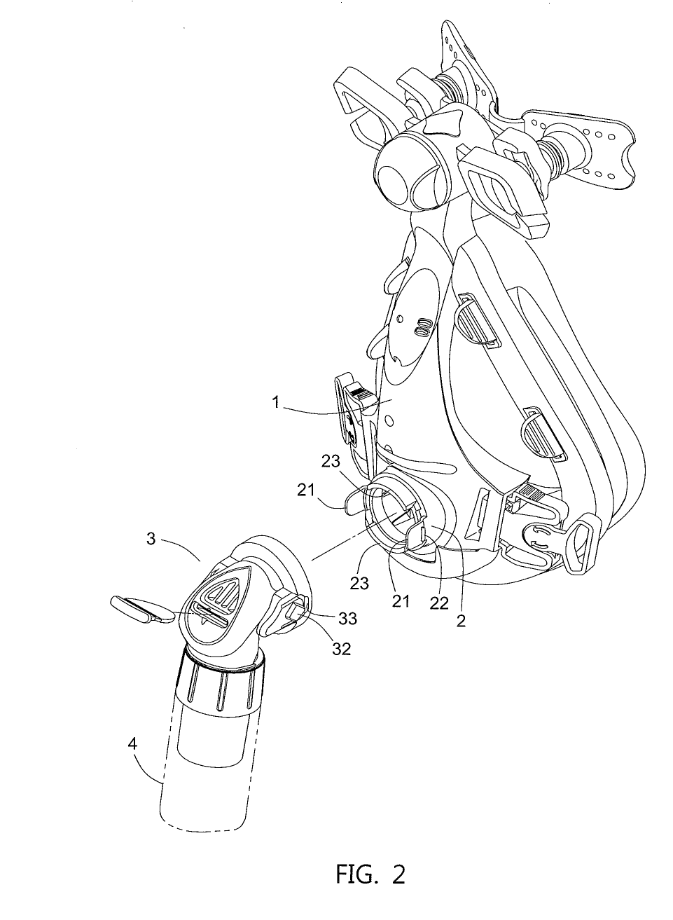

Please refer to FIG. 1 and FIG. 2. According to the present invention, a connecting member 2 is rotatably assembled to and secured on a front end of a respiratory mask 1. The connecting member 2 has two elastic, inward bendable tongues 21 extended from its two opposite outer edges. Each of the tongues 21 has a protuberance 22 formed on an outer surface thereof. The connecting member 2 further has two quasi-semicircular raised portions 23 formed at two other opposite outer edges thereof.

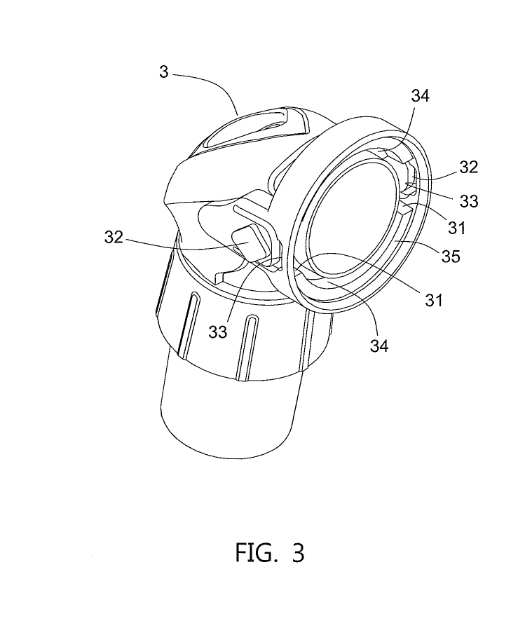

Please further refer to FIG. 3 now. A pipe-end joint 3 is configured to connect a gas delivery tube 4. Inside the pipe-end joint 3, there are two guiding stages 35 facing the connecting member 2. Between the ends of the guiding stages 35, two opposite sockets 31 are defined for fittingly receiving the two tongues 21, wherein the bottoms of the sockets 31 are more sunken into the pipe-end joint 3 as compared to the tops of the two guiding stages 35. In addition, the pipe-end joint 3 has a groove 34 for receiving the two raised portions 23 of the connecting member 2. Moreover, at the outer surface of the pipe-end joint 3, corresponding to the two sockets 31, there are two oppositely arranged resilient members 32. Each of the resilient members 32 has a free end that can resiliently retract inward the corresponding socket 31 when receiving a pressing force. The pipe-end joint 3 further has two retaining recesses 33 formed on the free ends of the resilient members 32 for retaining the protuberances 22 of the tongues 21.

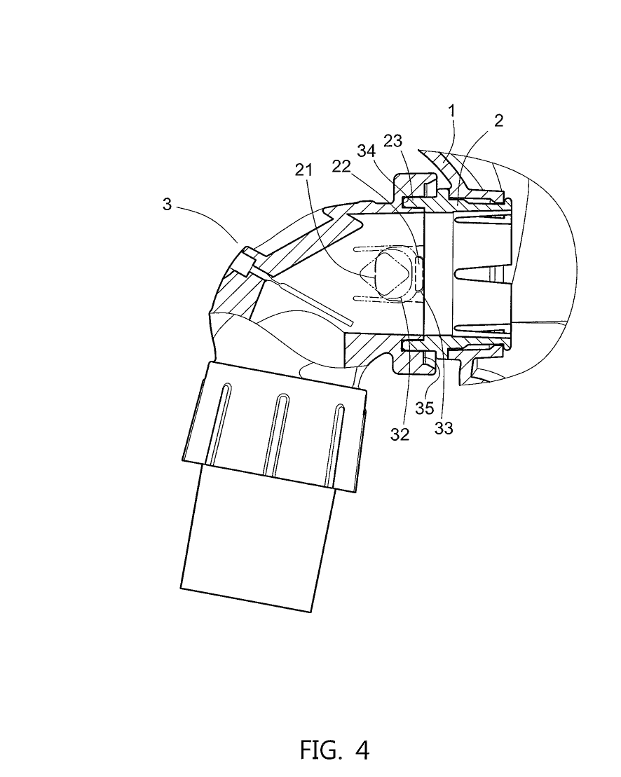

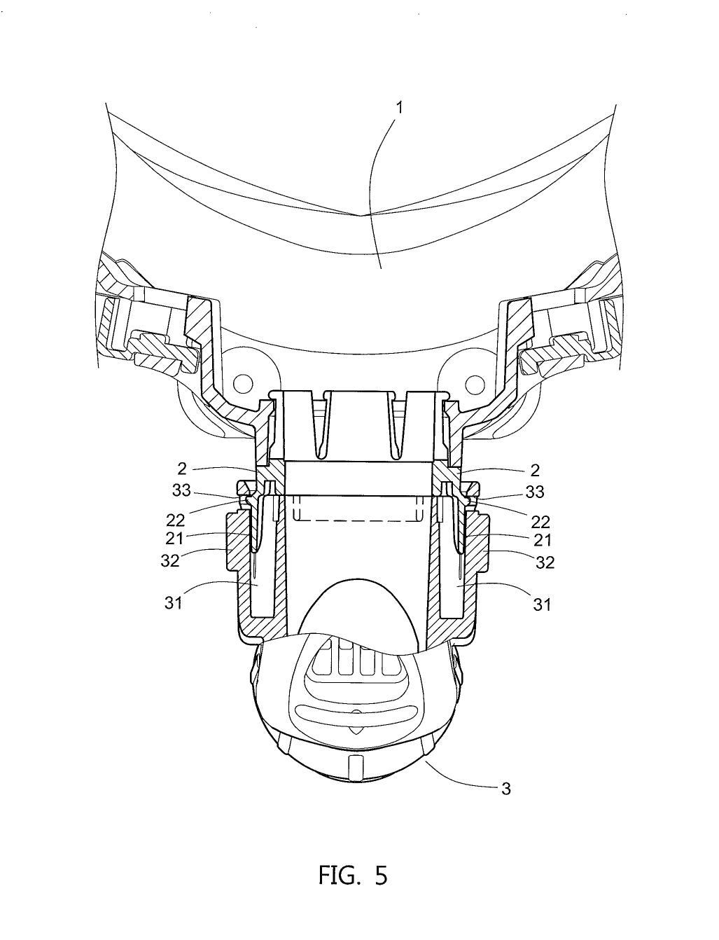

Accordingly, a shown in FIG. 4 and FIG. 5, when the pipe-end joint 3 is attached to the connecting member 2 on the respiratory mask 1, the tongues 21 of the connecting member 2 are inserted into the two sockets 31 of the pipe-end joint 3, and the raised portions 23 of the connecting member 2 are also received in the groove 34 of the pipe-end joint 3. In the event that the two sockets 31 and the two tongues 21 are not initially aligned well, the ends of the two tongues 21 abut against the guiding stages 35 inside the pipe-end joint 3. At this time, by simply rotate the pipe-end joint 3, the tongues 21 can move along the guiding stages 35 until falling into the sockets 31. After the tongues 21 are inserted into the sockets 31, the protuberances 22 on the tongues 21 resiliently engage with the retaining recesses 33 of the pipe-end joint 3, so that the pipe-end joint 3 is connected to the respiratory mask 1 through the connecting member 2. With the close engagement between the raised portions 23 of the connecting member 2 and the groove 34 inside the pipe-end joint 3, the combination of the connecting member 2 and the pipe-end joint 3 is secured to allow gas to smoothly flow along the gas delivery tube 4 and be guided into the respiratory mask 1. Thus, the connection between the pipe-end joint 3 and the connecting member 2 can be accomplished in a simple operation.

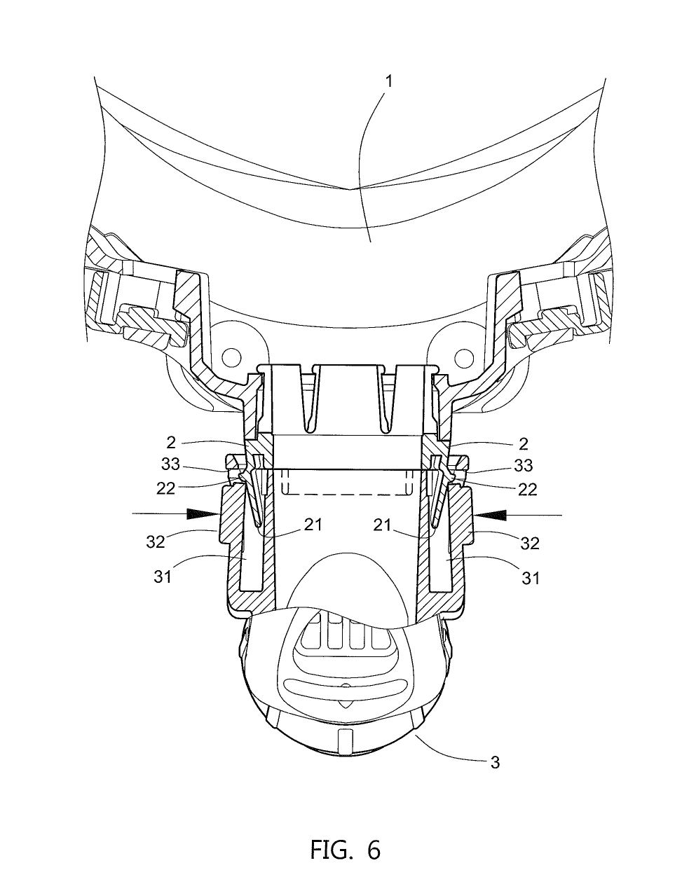

For detaching the pipe-end joint 3 from the respiratory mask 1, as shown in FIG. 6, a user may use his/her fingers to simultaneously, slightly press the resilient members 32 at the two sides of the pipe-end joint 3 inward to make the resilient members 32 sink and in turn move the two tongues 21 of the connecting member 2 inward. As a result, the protuberances 22 on the tongues 21 are withdrawn from the retaining recesses 33 of the pipe-end joint 3, and then the pipe-end joint 3 can be pulled outward and separate from the connecting member 2 smoothly. By doing this, the gas delivery tube 4 and the respiratory mask 1 can be easily separated.

Thereby, the quick-release coupling structure between the connecting member 2 on the respiratory mask 1 and the pipe-end joint 3 on the gas delivery tube 4 allows effort-saving and convenient attachment/detachment between the gas delivery tube 4 and the respiratory mask 1 for both professional and general users to easily separate and clean the respiratory mask 1 and the pipe-end joint 3.

What is claimed is:

1. A quick-release coupling structure between a gas delivery tube and a respiratory mask, the quick-release coupling structure comprising a connecting member that is rotatably assembled to a front end of the respiratory mask, and a pipe-end joint that is connected to the gas delivery tube and configured to be detachably coupled to the connecting member, wherein:
the connecting member having two elastic, bendable tongues provided at two opposite outer edges thereof, in which each said tongue has a protuberance formed on an outer surface thereof; and
the pipe-end joint having two sockets formed inside an end thereof facing the connecting member, in which the two sockets are configured to receive the two tongues, respectively, the pipe-end joint having two resilient members that are configured to press upon the tongues inserted in the sockets and are able to resiliently retract inward the sockets when receiving a pressing force, and the pipe-end joint further having two retaining recesses that are configured to retain the two protuberances on the two tongues that are inserted in the sockets, respectively.

2. The quick-release coupling structure of claim 1, wherein the connecting member further has two quasi-semicircular raised portions formed at two other opposite outer edges thereof, and the pipe-end joint correspondingly has a groove for receiving the two raised portions, respectively.

3. The quick-release coupling structure of claim 1, wherein the pipe-end joint has two guiding stages and the two sockets are formed between ends of the guiding stages so that the tongues that are not well aligned with the sockets are allowed to move along the guiding stages until falling into the sockets.
